(19) United States
(12) Patent Application Publication
(10) Pub. No.: US 2009/0062397 A1
(43) Pub. Date: Mar. 5, 2009

(54) COMPOUNDS AND METHODS FOR ENHANCING SOLUBILITY OF FLORFENICOL AND STRUCTURALLY-RELATED ANTIBIOTICS USING CYCLODEXTRINS

(75) Inventors: Serena Tongiani, Cranford, NJ (US); Keith Alan Freehauf, Stockton, NJ (US)

Correspondence Address: Intervet/Schering-Plough Animal Health;PATENT DEPARTMENT, PO BOX 318, 29160 Intervet Lane, MILLSBORO, DE, 19966-0318, US

(73) Assignee: Schering-Plough Animal Health Corporation

(21) Appl. No.: 12/108,032

(22) Filed: Apr. 23, 2008

Related U.S. Application Data
(60) Provisional application No. 60/914,376, filed on Apr. 27, 2007.

(51) Int. Cl. A61K 31/16

(52) U.S. Cl. 514/628

(57) ABSTRACT

The relatively low solubility of florfenicol (FEC) in water (1.3 mg/mL) limits its use in medicated drinking water systems for treatment of pulmonary disease of swine and poultry. Current formulations use a high volume organic solvent to reach the required FFC concentration of 13.5 mg/mL in an automated proportioner mixing tank system, with practical disadvantages for the users in the field. This invention relates to the effects of complex formation with natural and modified cyclodextrins on the aqueous solubility of FFC and antibiotics of related structure. Furthermore, this invention relates to the effects of polyethylene glycol (PEG-300) as a co-solvent in an FFC-cyclodextrin system to achieve the required FFC dose in the mixing tank system and to avoid high volumes of the organic solvent.

### BACKGROUND OF THE INVENTION

1. Field of the Invention

This invention relates to florfenicol-containing formulations having improved aqueous solubility. In particular, the invention relates to forming florfenicol complexes with cyclodextrins to improve its aqueous solubility.

2. Description of Related Art

Florfenicol is a broad spectrum antibiotic, structurally related to thiomphenicol and chloramphenicol developed as a veterinary treatment for use in cattle, swine, poultry, and fish.

Nuflor® Drinking Water Concentrate is a product of Schering-Plough designed to be an oral solution containing 23 mg/ml of florfenicol. This product was developed as an additive for drinking water systems for swine and poultry. When dosed at a concentration of 400 mg/gal (˜0.1 mg/ml) in the drinking water, the product minimizes mortality due to E. coli airsacculitis in broiler chickens and minimizes respiratory diseases in swine associated with Actinobacillus pleuropneumonia, P. multocida, Mycoplasma, Salmonella cholera suis and Streptococcus suis Type II.

One challenge associated with the Nuflor® product is the relatively low solubility of florfenicol in water. Typically, the product is either added directly to the bulk drinking water source or the product is added into the bulk drinking water source through a proportioner mixing tank system. These methods require the formulation to be soluble not only at the efficacious concentration of 400 mg/gal (˜0.1 mg/mL) in the bulk drinking water, but also at a concentration of ˜13.5 mg/mL to allow the use of a typical proportioner mixing ratio of 1:128 (1 oz. to 1 gallon). The solubility of florfenicol in water (1.23 mg/ml) allows for direct addition into the bulk drinking water with minimal problems; however, the required concentration for the proportioner systems is ten times greater. Therefore, efforts have been made to improve the aqueous solubility of florfenicol.

Cyclodextrins are a group of cyclic oligomers containing α-D-glucopyranose units linked with α-1-4 bonds. There are three well known naturally occurring CDs: α, β and γ, which are composed of six, seven or eight glucopyranose units, respectively (FIG. 1). In addition, modified CDs, such as HP-beta cyclodextrin and sulfoalkylether-cyclodextrin, which have the flexibility and ability to incorporate a variety of molecular frames are also known. Thanks to their peculiar structure, CDs are known to exhibit complex formation. Complex formation is defined as a reversible entrapment of a guest molecule into a host cavity to yield a new entity, i.e., the inclusion complex.

Cyclodextrins are used in pharmaceutical formulations because of their ability to increase the apparent solubility, stability and bioavailability of various medicinal agents by forming non-covalent inclusion complexes. Complexation with cyclodextrins is a useful way to enhance the solubility of poorly water soluble pharmaceutical compounds. Cyclodextrins are capable of forming inclusion complexes with organic and inorganic molecules in aqueous solutions acting as a host. The formation of inclusion complexes between the host cyclodextrin and the guest molecule is generally a function of the dimension of the cyclodextrin cavity and the dimension of the guest molecule. Natural cyclodextrins are somewhat limited in terms of size and shape, and modified cyclodextrins have therefore been employed to overcome the restrictions associated with natural cyclodextrins.

### SUMMARY OF THE INVENTION

The compositions of the invention are typically in the form of soluble powders, soluble granules, or lyophilized powders/cakes which are ready for reconstitution in a solvent. Other potential formulations include but are not limited to ready to use solutions, capsules, and tablets as well as other formulations useful for topical use.

In accordance therewith, one aspect of the invention provides compositions that contain:

a) from about 2.5 to about 35 wt % of florfenicol or a pharmaceutically acceptable salt thereof;

b) from about 0.5 to about 20 wt % of a cyclodextrin; and

c) from about 20 to about 95 wt % of water, a solvent and/or a mixture thereof.

The compositions can be in the form of an intimate complex of the florfenicol and the cyclodextrin by removing the water and/or solvent to form the complex.

The combination of the organic solvent and cyclodextrin has a significant effect in improving the solubility of florfenicol in water. Such synergism reduces the amount of solvent necessary to achieve the required concentration of the drug in solution and to maintain the drug in solution over time. This creates many advantages for practical use in the field, such as in automated proportioner mixing tank systems for animal drinking water systems. It also potentially provides a user-friendly florfenicol concentrate solution, thereby avoiding the use of high amounts of solvents and large volume containers which are difficult to handle and properly dispose. Furthermore, the combined action of cyclodextrin and the organic solvent in enhancing the florfenicol solubility may be used to reduce the amount of cyclodextrin in the formulation, thus limiting the overall cost of the product.

In addition, complex formation between the drug florfenicol and natural and/or modified cyclodextrins in water has been shown using solubility studies (FIG. 5 and FIG. 6) to have a calculated binding constant that is relatively high. This provides an advantage for the formulation of the drug florfenicol with cyclodextrin in water by enhancing its solubility, and providing a toll to reach the desired concentration in water without the co-aid of an organic solvent.

### DESCRIPTION OF THE DRAWINGS

FIG. 1 is a graphic representation of the chemical structure of α, β and γ cyclodextrin.

FIG. 2 is an illustration of the physical structure of cyclodextrin.

FIG. 3 is a graphic representation of the chemical structure of SBE- and HP-β-CD.

FIG. 4 is a graphic representation of the chemical structure of florfenicol.

FIG. 5 is a phase solubility diagram for florfenicol in β cyclodextrin.

FIG. 6 is a phase solubility diagram for florfenicol in γ cyclodextrin.

FIG. 7 is a phase solubility diagram for florfenicol in HP-β cyclodextrin.

FIG. 8 is a phase solubility diagram for florfenicol in Captisol (a type of sulfoalkylether cyclodextrin).

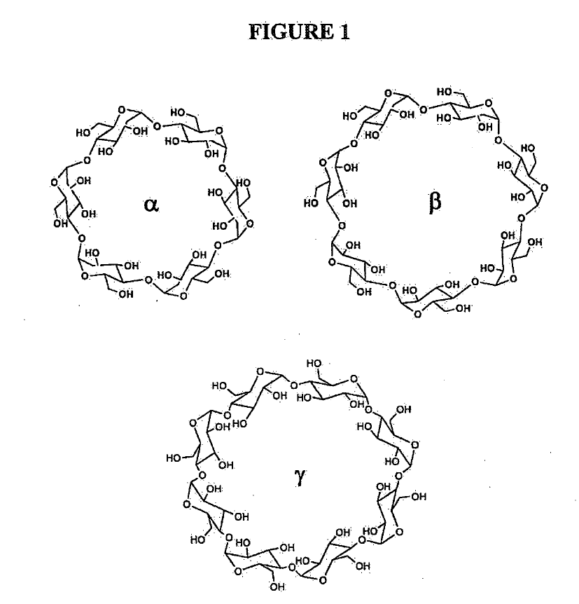

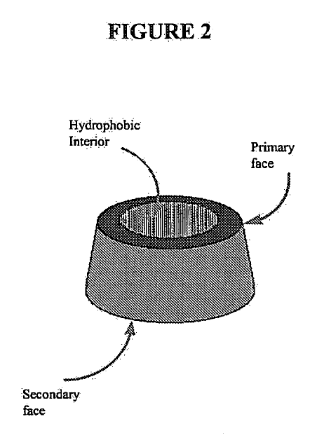

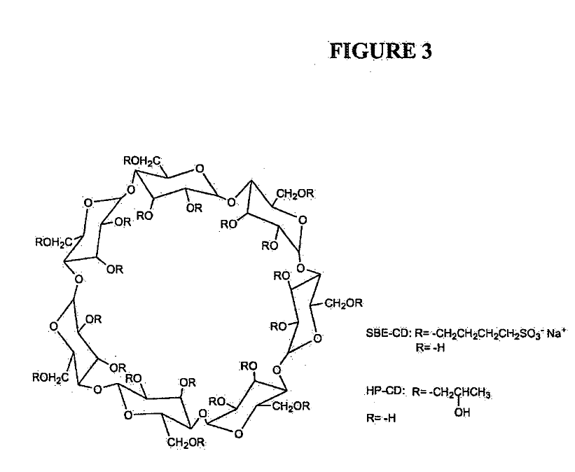

### DETAILED DESCRIPTION OF THE INVENTION

The invention provides compositions containing florfenicol or a pharmaceutically acceptable salt thereof for use in animal drinking water systems. In some preferred aspects of the invention, the florfenicol is present in an aqueous formulation, a solvent formulation, an aqueous/solvent formulation, a powder, granules or a lyophilized power/cake. Other formulations include ready to use solutions, capsules, tablets, as well as other formulations for topical use. Each of the above formulations can be added directly to the drinking water system to reach the antibiotic therapeutic dose with a fast solubility rate profile.

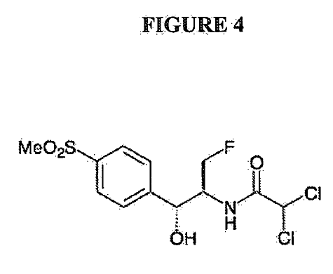

One of the key components of the formulations of the invention is the drug florfenicol. Florfenicol can be prepared as a free base or in its salt form and also in any of its derivative forms such us phosphate derivatives and any florfenicol pro-drugs. Florfenicol is not hygroscopic, so its incorporation in a formulation does not cause instability due to water absorption. Florfenicol is also known as [R-(R*,S*)]2,2-Dichloro-N-[1-(fluoromethyl)-2-hydroxy-2-[4-(methylsulfonyl)phenyl]-ethyl]acetamide (see FIG. 4). Processes for the manufacture of this preferred antibiotic, and intermediates useful in such processes, are described in U.S. Pat. Nos. 4,311,857; 4,582,918; 4,973,750; 4,876,352; 5,227,494; 4,743,700; 5,567,844; 5,105,009; 5,382,673; 5,352,832; and 5,663,361. Another preferred antibiotic is thiamphenicol. Pharmaceutically acceptable salts of the foregoing are also contemplated for addition to the formulations described herein.

In some aspects of the invention, the amount of florfenicol included in the compositions may range from about 2.5 to about 35 wt %. In preferred aspects, the amount of florfenicol is from about 15 to about 25 wt %, while in more preferred aspects, the amount is from about 20 to about 25 wt %.

The compositions of the invention preferably contain a cyclodextrin. The cyclodextrin can be a natural cyclodextrin, a modified cyclodextrin or a mixture thereof. A non-limiting list of natural cyclodextrins is α-cyclodextrin, β-cyclodextrin, γ-cyclodextrin and mixtures thereof. Modified cyclodextrins can include, for example, HP-beta cyclodextrin, sulfoalkyl-cyclodextrin, methylated cyclodextrin, ethylated cyclodextrin, and mixtures thereof.

The amount of cyclodextrin included in the compositions of the invention is from about 0.5 to about 20 wt % of the composition. Preferably, the amount is from about 0.5 to about 15 wt %, and more preferably from about 5 to about 10 wt % of the composition.

The compositions of the invention also preferably include water, a solvent, or a mixture thereof. The synergistic combination of a solvent and cyclodextrin significantly improves the solubility of florfenicol in water. Such synergism reduces the amount of solvent necessary to achieve the required concentration of the drug in solution and to maintain the drug in solution over time. As a result, it becomes easier to administer the drug through automated proportioner mixing tank systems for animal drinking water. In addition, a user-friendly florfenicol concentrate solution is achieved, thereby avoiding the use of high amounts of solvents and large volume containers which are difficult to handle and properly dispose. Furthermore, the combined action of cyclodextrin and the solvent in enhancing the florfenicol solubility may be used to reduce the amount of cyclodextrin in the formulation, thus limiting the overall cost of the product.

Non-limiting examples of solvents include polyethylene glycol 300, polyethylene glycol 400, propylene glycol, 2-pyrol, n-methylpyrol, and mixtures thereof.

The water is typically present in an amount of from about 20 to about 95 wt % of the composition. In a preferred embodiment, the water is present in an amount of from about 40 to about 80 wt % of the composition, and more preferably from about 5 to about 10 wt % of the composition.

Typically, the solvent is present in an amount of from about 20 to about 95 wt % of the composition. Preferably, the solvent is present in an amount of from about 40 to about 80 wt % of the composition, and more preferably from about 5 to about 10 wt % of the composition.

When the compositions of the invention contain a mixture of water and a solvent, the solvent:water ratio is typically in the range of from about 1 to about 10. Preferably, the ratio is from about 1 to about 5, and more preferably from about 1 to about 3.

Conventional excipients, such as colorings, fillers, diluents, surfactants, sweeteners, flavorings, preservatives, antioxidants, stabilizers, as well as other ancillary pharmaceutically acceptable ingredients and the like and mixtures thereof may be added to the formulations. For example, the formulations can also contains additional common excipients such us binders, lubricants, diluents, surfactants, solvents and mixtures thereof. One preferred diluent is lactose anhydrous. Other diluents that are suitable include without limitation microcrystalline cellulose, sorbitol, starch and calcium phosphate. The amount of diluent can range from about 0% by wt. to about 40% by wt. One preferred lubricant is magnesium stearate but other suitable lubricants can include, without limitation, calcium phosphate and/or calcium phosphate di-basic. The amount of lubricant can range from about 0% by wt. to about 5% by wt. One preferred surfactant is Tween80 but other suitable surfactants can include without limitation sodium lauryl sulfate. The amount of surfactant can range from about 0% by wt. to about 10% by wt. One preferred binder is polyvinylpyrrolidone (PVP) 30 in a range of between 2 and 20% by wt in aqueous or alcoholic solution. A non-limiting list of suitable alternatives may include polyvinylpyrrolidone 90, starch, methylcellulose, sodium carboxymethylcellulose, polyacrilamide and polyvinyl alcohols.

Other optional inert ingredients may be added to the present composition, as desired. Such ingredients include preservatives, antioxidants, stabilizers, colorings, sweeteners and flavorings. Exemplary preservatives include methyl p-hydroxybenzoate (methylparaben) and propyl p-hydroxybenzoate (propylparaben). Exemplary antioxidants include butylated hydroxyanisole and sodium monothioglycerol. Preferred stabilizers for use in the present invention include, for example, BHT or citric acid. A particularly preferred stabilizer to prevent degradation of any of the active ingredients in the formulations of the present invention is BHT in a concentration between 0.01% (w/w) and 0.05% (w/w). Other suitable stabilizers include, for example fumaric acid, malic acid, and tartaric acid. When a suitable acid is used as preservative it can be added in addition to or as part of the acid component, according with the stoichiometric ratio between acid and basic component in the effervescent formulation.

Exemplary sweeteners are mannitol, lactose, sucrose and dextrose.

In still further aspects of the invention, the compounds can contain a second pharmaceutically active composition that does not interfere or otherwise hamper the effectiveness of the florfenicol. It will be appreciated that other active ingredients may be combined with the formulations of the present invention. Such ingredients may include, for example, anti-inflammatory agents such as corticosteroids, NSAIDS, such as flunixin, COX-inhibitors and other analgesics, antiparasitic compounds such as, for example, an avermectin compound such as ivermectin, doramectin, milbemycin, selamectin, emamectin, eprinomectin, and moxidectin, and/or optionally a flukicide. It may also be preferred to employ a second antibiotic in the formulation. Preferred antibiotics may include tetracyclines. Particularly preferred is chlorotetracycline and oxytetracycline. Other preferred additional antibiotics include betalactams, such as penicillins, cephalosporins, e.g., penicillin, amoxicillin, or a combination of amoxicillin with clavulanic acid or other beta lactamase inhibitors, ceftiofur, cefquinome, etc. Also preferred antibiotics include fluoroquinolones, such as, for example, enrofloxacin, danofloxacin, difloxacin, orbifloxacin and marbofloxacin, and macrolide antibiotics such as tilmicosin, tulathromycin, erythromycin, azithromycin and pharmaceutically-acceptable salts there of and the like. Alternatively, one could include insect growth regulators in combination with the formulations of the present invention.

In another aspect of the invention, there are provided methods of treating or preventing diseases and florfenicol-susceptible conditions. The methods include introducing a sufficient amount of the composition described herein into water, and administering the resultant solution to a subject in need thereof, as part of the liquid to be ingested by the subject, e.g., the formulation may be added into its drinking water system to administer the treatment and therapeutic dose to livestock.

The amount administered is a therapeutically or prophylactically-effective amount of the florfenicol solution resulting from the introduction of the compound into water. In most aspects of this embodiment, the amount of compound added to water is an amount that is sufficient to bring the concentration of florfenicol in the drinking water to from about 0.01 mg/mL to about 0.2 mg/mL. Preferably the concentration will be about 0.1±0.09 mg/mL in the bulk drinking water, and a concentration of about 13.5±0.1 mg/mL when the aqueous solutions are used in a typical proportioner mixing ratio of 1:128 gallons. Depending upon the condition being treated and the type, size, weight, etc. of the animal being treated, it is contemplated that suitable periods of treatment will range from about 1 to about 5 days or longer if needed using the novel compounds in drinking water at the concentrations mentioned above. As will be appreciated by those of ordinary skill, the animals will drink the treated water ad libitum. It is nonetheless contemplated that sufficient amounts of the florfenicol will be administered to the animals in need thereof when it is available for drinking for the periods mentioned above.

The compounds of the present invention may, if desired, be presented in a pack or dispenser device, such as an FDA approved kit, which may contain one or more unit dosage forms containing the compound in the form of compressed tablets, granules or a lyophilized powder/cake containing the active ingredient. The pack may, for example, comprise metal or plastic foil, such as a blister pack. The pack may also consist of a soluble biodegradable pouch ready to use, sealed in a metal plastic foil. The pack or dispenser device may be accompanied by instructions for administration. The pack or dispenser may also be accompanied by a notice associated with the container in a form prescribed by a governmental agency regulating the manufacture, use or sale of pharmaceuticals, which notice is reflective of approval by the agency of the form of the compositions or of human or veterinary administration. Such notice, for example, may be of the labeling approved by the U.S. Food and Drug Administration for prescription drugs or of an approved product insert. Compositions comprising a compound of the invention formulated in a compatible pharmaceutical carrier may also be prepared, placed in an appropriate container, and labeled for treatment of an indicated condition. Thus, the kit can be used in connection with the treating or preventing of a bacterial infection or other disease in a subject in need thereof and include a sufficient amount of the compound described herein and instructions for introducing the compound into drinking water to be given to the subject in need thereof.

### EXAMPLES

The following examples are provided to illustrate certain embodiments of this invention and are not intended, nor are they to be construed, to limit its scope in any manner whatsoever.

Phase solubility analysis is one of the methods used to determine a drug:CD binding constant and its consequences on the drug solubility have been extensively described in the literature by Higuchi and Connors. In such experiments, the solubility of a substance (S) is monitored with increases in the concentration of a ligand (L) (cyclodextrin in this case). The experiments are conducted in a series of tubes or vials containing the same volume of ligand solutions with increasing concentrations, except that one tube contains the solvent only. To each tube is added a known amount of the substance or drug, and the samples are equilibrated at constant temperature. The solution phase is then analyzed for the total substance concentration. If a soluble complex forms, the substance concentration should vary with increasing ligand concentration. The solubility behavior can be determined using a phase diagram by plotting the total substance concentration versus the total ligand concentration.

Florfenicol solubility in mixed solvents, such as water/PEG-300, and florfenicol complexes with beta cyclodextrin were evaluated by solubility studies using High Performance Liquid Chromatography (HPLC). The HPLC system consisted of a Waters Alliance Separation Module equipped with a Waters 2996 Photodiode Array Detector interfaced with Millennium Chromatography manager data software. The HPLC mobile phase consisted of acetonitrile and a 0.1% phosphoric acid aqueous solution (30:70 v/v). A Phenomenx®, Luna C8 5 micron column was used. The flow rate was 1.0 mL/min and the detection wavelength was 260 nm. Binding constants were calculated using the Higuchi Connors method from solubility study plots. The solubility of florfenicol in aqueous CD/PEG-300 systems was also investigated with the same technique.

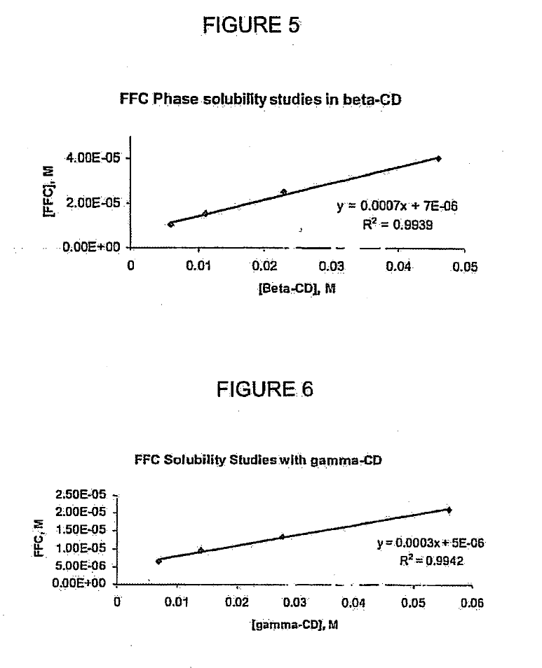

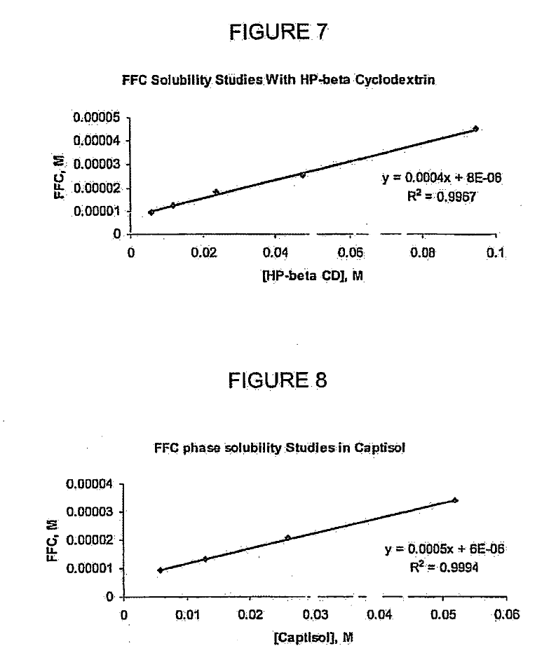

In a first set of experiments, phase solubility studies were conducted for FFC in natural and modified cyclodextrins. The results of these studies are reported in Tables I, II, III, IV and V below, and the phase solubility diagrams are reported in FIGS. 5, 6, 7 and 8.

TABLE I
Experimental water solubility for drug FFC:

| | Solubility of FFC, mg/mL |
|---|---|
| Standard 1 | 1.91 |
| Standard 2 | 1.90 |
| Standard 3 | 1.95 |

The calculated FFC Solubility S₀ was 4.90E-07 M.

TABLE II
FFC Solubility in Beta CD Complex:

| Beta-CD, M | Average FFC, mg/mL | STDEV | FFC, M |
|---|---|---|---|
| 0.05 | 15.59 | 0.66 | 4.04E−05 |
| 0.02 | 9.79 | 0.02 | 2.54E−05 |
| 0.01 | 5.91 | 0.17 | 1.53E−05 |
| 0.006 | 4.03 | 0.01 | 1.045E−05 |

The calculated binding constant for FFC:CD complex was 1429.57 M⁻¹.

TABLE III
FFC Solubility in Gamma CD Complex:

| Gamma-CD, M | Average FFC, mg/mL | STDEV | FFC, M |
|---|---|---|---|
| 0.11 | 10.77 | 0.30 | 2.79E−05 |
| 0.06 | 8.20 | 0.45 | 2.12E−05 |
| 0.03 | 5.24 | 0.26 | 1.36E−05 |
| 0.014 | 3.70 | 0.01 | 9.60E−06 |
| 0.007 | 2.51 | 0.46 | 6.51E−6 |

The calculated binding constant for FFC:Gamma-CD Complex was 612.43 M⁻¹.

TABLE IV
FFC Solubility in Hp-beta CD Complex:

| HP-Beta-CD, M | Average FFC, mg/mL | STDEV | FFC, M |
|---|---|---|---|
| 0.09 | 17.43 | 0.64 | 4.52E−05 |
| 0.05 | 9.76 | 0.49 | 2.53E−05 |
| 0.02 | 7.00 | 0.01 | 1.82E−05 |
| 0.012 | 4.88 | 0.27 | 1.26E−5 |
| 0.006 | 3.72 | 0.023 | 9.62E−6 |

The calculated binding constant for FFC:HP-CD complex was 816.652 M⁻¹.

TABLE V
FFC Solubility in Captisol ® Complex:

| Captisol ®, M | Average FFC, mg/mL | STDEV | FFC, M |
|---|---|---|---|
| 0.10 | 19.18 | 0.73 | 4.97E−05 |
| 0.05 | 13.17 | 0.21 | 3.41E−05 |
| 0.03 | 7.98 | 0.23 | 2.07−05 |
| 0.013 | 5.11 | 0.03 | 1.32E−5 |
| 0.006 | 4.92 | 0.31 | 9.28E−6 |

The calculated binding constant for FFC:Captisol® complex was 1020.92 M⁻¹.

In a second set of experiments, the effects of different percentages of PEG-300 when used as a co-solvent were investigated in a FFC-cyclodextrin complex system.

The results obtained for the FEC-CD complex in the presence of different percentages of PEG-300 and different molar concentrations of cyclodextrin are reported in Tables VI and VII below.

TABLE VI
Effect of different percentage of PEG-300
on a FFC: 10 mM Beta-CD system:

| Beta-CD: PEG-300 System | Solubility of FFC, mg/mL |
|---|---|
| 10 mM beta/3% PEG-300 | 7.51 |
| 10 mM beta/10% PEG-300 | 6.31 |
| 10 mM beta/50% PEG-300 | 12.28 |

TABLE VII
Effect of different percentage of PEG-300
on a FFC: 50 mM Beta-CD system:

| Beta-CD: PEG-300 System | Solubility of FFC, mg/mL |
|---|---|
| 50 mM beta/20% PEG-300 | 10.57 |
| 50 mM beta/30% PEG-300 | 14.36 |
| 50 mM beta/50% PEG-300 | 15.50 |

Based on the solubility results obtained for FFC, it was demonstrated that complexation with cyclodextrin is an alternative to the use of high percentage organic solvents for the formulation of a concentrated solution of florfenicol (and related-structure antibiotics) to be used in drinking water systems. Furthermore, when PEG-300 was used as co-solvent for a FFC:cyclodextrin complex, the solubility of FFC was greatly increased.

The complex formation between cyclodextrin and a drug such as florfenicol may be achieved using different techniques. In one technique, the complex can be formed in a water solution. For instance, a saturated water solution of the drug is added to 10% cyclodextrin and incubated for 24 hours. The excess drug is removed and the solvent may be added at this point. In a second technique, the complex is formed by a “paste technique” using a minimal amount of solvent, and the paste of cyclodextrin and the drug is at added to the formulation. In a third technique, the cyclodextrin/drug complex is formed directly in the solvent under continuous agitation.

Non-limiting examples of possible formulations for use in drinking water systems are:

### Example 1

| Component | % (w/w) |
|---|---|
| Florfenicol | 1.5% |
| HP-Beta CD | 10% |
| Water | q.s |

The formulation reported in Example 1 was a clear solution, or in alternative, it could be lyophilized to be presented as a powder to be reconstituted in water.

### Example 2

| Component | % (w/w) |
|---|---|
| Florfenicol | 4.5% |
| HP-Beta CD | 30% |
| Water | q.s |

The formulation reported in Example 2 was a clear solution, or in alternative, it could be lyophilized to be presented as a powder to be reconstituted in water.

### Example 3

| Component | % (w/w) |
|---|---|
| Florfenicol | 2% |
| Beta CD | 20% |
| PEG-300 | 30% |
| Water | q.s |

The formulation reported in Example 3 was a clear solution, or in alternative, it could be lyophilized to be presented as a powder to be reconstituted in water.

With FFC:CD complexation, it is possible to achieve desirable FFC solubility, to have the desired concentration in the automated proportioner mixing tank system, and to maintain the drug in solution over time. Furthermore, samples containing multiple solubilizing agents, such as beta-CD and PEG-300, show a significant increase in FCC solubility in water. The synergism of cyclodextrin solutions with PEG-300 reduced the amount of solvent (polyethylene glycol) necessary to achieve the required concentration in the automated proportioner mixing tank system. The synergistic combination of cyclodextrin with PEG-300 also provides a user-friendly FFC concentrate solution, thereby avoiding the use of high amounts of solvents and large volume containers which are difficult to handle and dispose of properly.

Although certain presently preferred embodiments of the invention have been described herein, it will be apparent to those skilled in the art to which the invention pertains that variations and modifications of the described embodiments may be made without departing from the spirit and scope of the invention.

Accordingly, it is intended that the invention be limited only to the extent required by the appended claims and the applicable rules of law.

We claim:

1. A composition comprising:
a) from about 2.5 to about 35 wt % of florfenicol or a pharmaceutically acceptable salt thereof;
b) from about 0.5 to about 20 wt % of a cyclodextrin; and
c) from about 20 to about 95 wt % of water, a solvent and/or a mixture thereof.

2. The composition of claim 1, wherein the florfenicol or pharmaceutically acceptable salt thereof is from about 15 to about 25 wt % of the composition.

3. The composition of claim 2, wherein the florfenicol or pharmaceutically acceptable salt thereof is from about 20 to about 25 wt % of the composition.

4. The composition of claim 1, wherein the cyclodextrin is a natural cyclodextrin, a modified cyclodextrin, or a mixture thereof.

5. The composition of claim 4, wherein the natural cyclodextrin is selected from the group consisting of α-cyclodextrin, β-cyclodextrin, γ-cyclodextrin, and mixtures thereof.

6. The composition of claim 4, wherein the modified cyclodextrin is selected from the group consisting of HP-beta cyclodextrin, sulfoalkyl-cyclodextrin, methylated cyclodextrin, ethylated cyclodextrin, and mixtures thereof.

7. The composition of claim 1, wherein the cyclodextrin is from about 0.5 to about 15 wt % of the composition.

8. The composition of claim 7, wherein the cyclodextrin is from about 5 to about 10 wt % of the composition.

9. The composition of claim 1, wherein the water is from about 40 to about 80 wt % of the composition.

10. The composition of claim 9, wherein the water is from about 5 to about 10 wt % of the composition.

11. The composition of claim 1, wherein the solvent is selected from the group consisting of polyethylene glycol 300, polyethylene glycol 400, propylene glycol, 2-pyrol, n-methylpyrol and mixtures thereof.

12. The composition of claim 1, wherein the solvent is from about 10 to about 60 wt % of the composition.

13. The composition of claim 12, wherein the solvent is from about 15 to about 40 wt % of the composition.

14. The composition of claim 1, further comprising a member of the group consisting of preservatives, antioxidants, stabilizers, colorants, sweeteners, flavorants, and mixtures thereof.

15. A method of treating or preventing a disease, comprising:
introducing the composition of claim 1 directly into water or into water through a proportioner mixing tank system; and
administering to a subject in need thereof a therapeutically effective amount of a product resulting from the introduction of the composition into the water.

16. The method of claim 15, wherein the concentration of florfenicol or pharmaceutically acceptable salt thereof administered to said subject is from about 0.01 to about 0.2 mg/ml.

17. A kit for treating or preventing a disease in a subject, comprising the composition of claim 1 and instructions for introducing the composition into drinking water given to the subject.

18. A florfenicol complex comprising an intimate combination of florfenicol and cyclodextrin prepared by forming the composition of claim 1 and removing the water, solvent and/or mixture thereof to form the complex.

19. The composition of claim 1, wherein the solvent:water ratio is from about 1 to about 10.

20. The composition of claim 19, wherein the solvent:water ratio is from about 1 to about 5.

21. The composition of claim 20, wherein the solvent:water ratio is from about 1 to about 3.
